(19) United States
(12) Patent Application Publication
(10) Pub. No.: US 2003/0133906 A1
(43) Pub. Date: Jul. 17, 2003

(54) Method for treating and preventing pancreatitis

(75) Inventors: Jacques Deviere, Genappe (BE); Marielle Cohard, Summit, NJ (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(73) Assignee: Schering Corporation

(21) Appl. No.: 10/304,940

(22) Filed: Nov. 26, 2002

Related U.S. Application Data
(60) Provisional application No. 60/333,983, filed on Nov. 28, 2001.

(51) Int. Cl. A61K 38/20

(52) U.S. Cl. 424/85.2

(57) ABSTRACT

This invention relates to the use of interleukin-10 (IL-10) for the prevention and treatment of pancreatitis. It provides methods for preventing the onset or worsening of pancreatitis in patients at risk of developing such condition by administering a therapeutically effective amount of IL-10. In a specific embodiment, IL-10 is administered to patients at risk of developing pancreatitis due to a procedure such as endoscopic retrograde pancreotography.

### CROSS-REFERENCE TO RELATED APPLICATIONS

This application is a non-provisional application that claims priority under 35 U.S.C. § 119(e) of provisional application, U.S. Ser. No. 60/333,983 filed Nov. 28, 2001, the contents of which are hereby incorporated by reference in their entireties.

### FIELD OF THE INVENTION

This invention relates to the use of interleukin-10 (IL-10) for the prevention and treatment of pancreatitis, and the like.

### BACKGROUND OF THE INVENTION

Acute pancreatitis is a major complication of endoscopic retrograde cholangiopancreatography (ERCP). Unlike hemorrhage, duodenal perforation, or cholangitis, the incidence of pancreatitis has not decreased with the technical improvements of recent years and expertise of the operators [Freeman et al., N. Engl. J. Med. 335:909-918 (1996); Huibregtse K, N. Engl. J. Med. 335:961-963 (1996)]. The risk of post-ERCP pancreatitis varies greatly with the indications, being <5% for management of common bile duct (CBD) stones and reaching 20% or more in cases of suspected sphincter of Oddi dysfunction (SOD) and manipulation of small bile ducts or of a well-functioning pancreatic gland in young patients [Freeman et al., supra; Sherman et al., Gastroenterology 101:1068-1075 (1991); Sherman et al., Gastrointest. Endosc. 36:462-466 (1990); Messmann et al., Gut 40:80-85 (1997); Loperfido et al., Gastrointest. Endosc. 48:1-10 (1998)].

A silent increase in serum pancreatic enzyme levels is much more frequent than clinical pancreatitis after ERCP and may be encountered in up to 70% of the cases [Messmann et al., supra; Poon et al., Gastrointest. Endosc. 49:593-598 (1999); Andriulli et al., Gastrointest. Endosc. 51:1-7 (2000); Cavallini et al., N. Engl. J. Med. 335:919-923 (1996)].

Among the numerous drugs that have been tested to prevent post-ERCP pancreatitis, only gabexate mesylate [a synthetic protease inhibitor; Cavallini et al., N. Engl. J. Med. 335:919-923 (1996)], and native somatostatin [an inhibitor of pancreatic enzymes secretion; Poon et al., Gastrointest. Endosc. 49:593-598 (1999); Andriulli et al., Gastrointest. Endosc. 51:1-7 (2000)] have proven effective. However, their short half-lives make continuous infusion lasting for 6-12 hours necessary, which further increases the cost of the procedure and renders outpatient endotherapy difficult [Freeman et al., Gastrointest. Endosc. 49:580-586 (1999); Ho et al., Gastrointest. Endosc. 49:587-592 (1999)]. Whatever the cause of pancreatitis, initial intracellular events are followed by an early local and systemic inflammatory reaction that is boosted by chemokines and proinflammatory cytokines [Saluja et al., Digestion 60:27-33 (1999); Norman et al., Digestion 60:57-60 (1999)].

The citation of any reference herein should not be construed as an admission that such reference is available as “prior art” to the instant application.

### SUMMARY OF THE INVENTION

The present invention discloses that IL-10 is able to limit the proinflammatory cascade that plays a major role in the development of pancreatic necrosis and distant organ damage. Moreover, the present invention discloses that post-ERCP pancreatitis is a unique model for studying the potential role of prophylactic IL-10 administration in reducing the incidence and/or the severity of human pancreatitis, since the initial noxious event is timely well defined.

The present invention relates generally to manipulation of cytokine synthesis during pancreatitis and specifically to using an anti-inflammatory cytokine, IL-10, to treat and prevent pancreatitis. The present invention provides methods for preventing the onset or worsening of pancreatitis in patients at risk of developing such condition by administering a therapeutically effective amount of IL-10.

Thus, in one embodiment, the invention provides a method of preventing pancreatitis in a patient at risk for developing such condition due to a pancreatitis-inducing event by administering a therapeutically effective amount of IL-10 before the onset of the pancreatitis.

In another embodiment, the invention provides a method of preventing exacerbation of pancreatitis in a patient at such risk due to a pancreatitis-inducing event by administering to the patient a therapeutically effective amount of IL-10 before the pancreatitis.

In specific embodiments of a method of the invention, IL-10 is administered to patients at risk of developing pancreatitis due to a procedure such as endoscopic retrograde pancreotography. Other causes for risk of developing pancreatitis, which are avoided by a method of the invention, include biliary tract disease, alcohol, drug therapies (e.g., azathioprine, sulfasalazine, furosemide, valproic acid), vaccination against infectious disease, estrogen use, infection (e.g., mumps), hypertriglyceridemia, ERCP, percutaneous transhepatic cholangiography, structural abnormalities of the pancreatic duct (e.g., stricture, cancer, pancreas divisum), structural abnormalities of the common bile duct and ampullary region (e.g., choledochal cyst, sphincter of Oddi stenosis), hemobilia, bile duct obstruction, surgery (e.g., stomach, biliary tract and coronary artery bypass grafting), vascular disease (e.g. severe hypotension), blunt or penetrating trauma, hyperparathyroidism and hypercalcemia, renal transplantation, hereditary pancreatitis, peritoneal dialysis, and cigarette smoking.

### DETAILED DESCRIPTION OF THE INVENTION

IL-10 used in this invention can be obtained from various sources known in the art. Preferably, the IL-10 is of mammalian origin, e.g. human or mouse, and human IL-10 (hIL-10) is used for treatment of humans. The IL-10 can be recombinantly or chemically prepared according to conventional methods, [e.g., see U.S. Pat. Nos. 5,231,012 and 6,217,857, the contents of which are hereby incorporated by reference in their entireties] for the preparation of human and mouse IL-10. In another embodiment, the IL-10 can be viral IL-10, e.g., viral IL-10 from Epstein Barr virus (BCRF1 protein), which is described in, e.g., Moore et al., Science 248:1230 (1990). Recombinant human IL-10 is also commercially available, e.g., from PeproTech, Inc., Rocky Hill, N.J.

An IL-10 protein used in the present invention can be modified (described herein as a “variant”) that (1) contains an amino acid sequence which shares an observed homology of at least 70%, preferably at least 80%, more preferably at least 90%, and most preferably at least 95%, with the sequence of a mature native IL-10 protein, i.e., lacking any leader sequences, as described in U.S. Pat. Nos. 5,231,012 or 6,217,857, the contents of which are hereby incorporated by reference in their entireties, and (2) has a biological activity that is common to native IL-10, e.g., inhibition or substantial reduction of serum levels of IL-2, lymphotoxin, IL-3, and/or GM-CSF, or inhibition of cytokine production, e.g., IL-1, IL-6, and TNF-α, by activated macrophages. Assays to determine IL-10 activity are generally known in the art [e.g., see U.S. Pat. Nos. 5,231,012 and 6,217,857, the contents of which are hereby incorporated by reference in their entireties].

Amino acid sequence homology, or sequence identity, is determined by optimizing residue matches and, if necessary, by introducing gaps as required. Homologous amino acid sequences are typically intended to include natural allelic, polymorphic and interspecies variations in each respective sequence. See Needleham et al., J. Mol. Biol. 48:443-453 (1970); Sankoff et al. in Time Warps, String Edits, and Macromolecules: The Theory and Practice of Sequence Comparison, 1983, Addison-Wesley, Reading, Mass.; and software packages from IntelliGenetics, Mountain View, Calif., and the University of Wisconsin Genetics Computer Group, Madison, Wis.

A modified IL-10 (i.e. variant) for use in the present can vary from the naturally-occurring IL-10 sequence at the primary structure level, e.g., by containing amino acid insertions, substitutions, deletions and fusions. Preferably, amino acid substitutions are conservative, e.g., basic residues are replaced with other basic residues, acidic residues are replaced with other acidic residues, etc. Such modifications can be made in a number of combinations, using various conventional techniques known in the art, to produce a final modified IL-10 variant.

IL-10 variants can be prepared with various objectives in mind, including increasing serum half-life, reducing an immune response against the IL-10, facilitating purification or preparation, decreasing conversion of IL-10 into its monomeric subunits, improving therapeutic efficacy, and lessening the severity or occurrence of side effects during therapeutic use. The amino acid sequence variants are typically predetermined variants not found in nature, although others may be post-translational variants, e.g., glycosylated variants or pegylated variants as described in U.S. patent application Ser. No. 09/967,223 filed on Sep. 28, 2001 in the name of Schering Corporation, the contents of which are hereby incorporated by reference in their entireties.

To prepare pharmaceutical compositions containing IL-10 for use in a method of this invention, the IL-10 can be admixed with a pharmaceutically acceptable carrier or excipient, which are preferably inert. A pharmaceutical carrier can be any compatible non-toxic substance suitable for delivery of the polypeptide to a patient. Preparation of such pharmaceutical compositions is known in the art. See, e.g., Remington's Pharmaceutical Sciences, 18th ed., 1990, Mack Publishing Co., Easton, Pa. and U.S. Pharmacopeia: National Formulary, 1984, Mack Publishing Company, Easton, Pa.

Pharmaceutical compositions containing IL-10 useful for parenteral administration can be prepared according to conventional methods known in the art, e.g., see, Remington's Pharmaceutical Science. When administered parenterally, the IL-10 is formulated in a unit dosage injectable form (solution, suspension, emulsion) in association with the pharmaceutical carrier, e.g., normal saline, Ringer's solution, dextrose solution, Hank's solution, or non aqueous carriers such as fixed oils and ethyl oleate. A carrier protein, e.g., human serum albumin, can also be used if desired. A preferred carrier is 5% dextrose/saline. The carrier may contain minor amounts of additives such as substances that enhance isotonicity and chemical stability, e.g., buffers and preservatives. For parenteral administration, the IL-10 is typically provided as a lyophilized powder, which is reconstituted with sterile water for injection. The powder may contain minor amounts of additives such as substances that enhance isotonicity and chemical stability, e.g., buffers and preservatives. The IL-10 is preferably formulated in purified form substantially free of aggregates and other source proteins at concentration ranges as described herein.

The proportion of IL-10 and an additive can be varied over a broad range so long as both are present in therapeutically effective amounts. Thus, on a per-dose basis, the amount of the IL-10 can range from about 0.1 to 100 μg/kg body weight. Preferably, the amount of IL-10 is from about 1 to 50 μg/kg body weight. Still, more preferably, the amount of IL-10 is from about 2.5 μg to about 25 μg/kg body weight.

As used herein, the phrase “therapeutically effective amount” means an amount sufficient to prevent onset or exacerbation (i.e. worsening) of a pancreatitis condtion [i.e., prevent or improve a symptom, sign or marker of a pancreatitis condition, which include, e.g., abdominal pain which may radiate to the back, hyperhydrolesemia (describe infra), elevated serum levels of amylase, isoamylase, lipase, C-reactive protein, trypsinogen-2, trypsin, phospholipase A₂, and/or trypsin-2-alpha 1 antitrypsin complex]. Practitioners having ordinary skill in the art can evaluate a patient for such symptoms and markers of pancreatitis. Scoring systems used to diagnose pancreatitis in a patient, e.g., Ranson, Osborne and Apache II, and/or visualization technologies, e.g., ultrasonography, CT scan, and ERCP, can also be used.

The term “prevent”, and grammatical variations thereof, as used herein, means reduce or avert a symptom, sign or marker associated with a pancreatitis condition or with a pancreatitis-inducing event which can cause pancreatitis. Administration of IL-10 to such individuals at high risk for developing pancreatitis prior to the onset of these symptoms, signs or markers would avert such symptoms, thus preventing the onset of the disease.

Administration of IL-10 is preferably parenteral, e.g., by injection through some route other than the alimentary canal, including subcutaneous, intramuscular, intraorbital, intracapsular, intraspinal, intrasternal, intraperitoneal, intravenous, infusion or by another acceptable systemic method. Administration by subcutaneous injection is most preferred. Alternatively, the IL-10 may be administered by an implantable or injectable drug delivery system. See, e.g., Urquhart et al., Ann Rev. Pharmacol. Toxicol 24:199 (1984); Lewis, ed., 1981, Controlled Release of Pesticides and Pharmaceuticals, Plenum Press, New York, N.Y.; U.S. Pat. Nos. 3,773,919, and 3,270,960, the contents of which are hereby incorporated by reference in their entireties. If oral administration is desirable then formulations known in the art, which protect the IL-10 from gastrointestinal proteases, can be employed. IL-10 can also be delivered by standard gene therapy techniques, including e.g., direct DNA injection into tissues, the use of recombinant viral vectors or phospholipid and implantation of transfected cells. See, e.g., Rosenberg, J. Clin. Oncol. 10:180 (1992).

IL-10 can be administered alone or in combination (“co-administered”) with one or more of the other agents, such as gabexate mesylate and somatostatin, commonly used to prevent or ameliorate the symptoms of pancreatitis in a method of the present invention. The co-administration can be sequential or simultaneous. “Co-administration” generally means that the multiple (two or more) therapeutics are present in the recipient during a specified time interval. Typically, if a second agent is administered within the half-life of the first agent, the two agents are considered co-administered.

The present invention provides a method for preventing pancreatitis in an individual at risk for developing pancreatitis (e.g., due to a pancreatitis-inducing event such as a medical procedure, therapy, preexisting condition, disease, etc.) by administering a therapeutically effective amount of IL-10. The invention further provides a method for preventing the exacerbation of an existing pancreatitis condition due to a pancreatitis-inducing event, by administering a therapeutically effective amount of IL-10.

The pancreatitis-inducing event is any circumstance that predisposes or places an individual at risk for developing pancreatitis or worsens an existing pancreatitis condition, including, but not be limited to, biliary tract disease, alcohol, drug therapies (e.g., azathioprine, sulfasalazine, furosemide, valproic acid), vaccination against infectious disease, estrogen use (e.g., associated with hyperlipidemia), infection (e.g., mumps), hypertriglyceridemia, ERCP, percutaneous transhepatic cholangiography (PTC), structural abnormalities of the pancreatic duct (e.g., stricture, cancer, pancreas divisum), structural abnormalities of the common bile duct and ampullary region (e.g., choledochal cyst, sphincter of Oddi stenosis), hemobilia (e.g., as caused by liver biopsy), bile duct obstruction, surgery (e.g., stomach, biliary tract and coronary artery bypass grafting), vascular disease (especially severe hypotension), blunt or penetrating trauma, hyperparathyroidism and hypercalcemia, renal transplantation, hereditary pancreatitis, peritoneal dialysis, cigarette smoking, and other causes reported in the art.

For example, a patient diagnosed with having gallstones may undergo therapeutic ERCP, which predisposes the patient to pancreatitis or could exacerbate an existing acute pancreatitis condition leading to death. IL-10 administered according to a method of the invention is contemplated as a prophylaxis against both the onset of acute pancreatitis and the worsening of the pancreatitis condition due to the therapeutic ERCP.

To prevent onset of pancreatitis or prevent exacerbation of a pre-existing condition of pancreatitis due to a pancreatitis-inducing event, an IL-10 is administered before the onset or exacerbation occurrs and preferably, although not required, before the event, e.g., within about 24 to 48 hours thereof. Preferably, the IL-10 is administered within about 12 hours of the event, more preferably within about 4 hours of the event, more preferably still within 1 hour of the event and most preferably about 15 to 30 minutes of the event. If it is desirable to administer the IL-10 after the pancreatitis-inducing event, it is preferably administered before the onset of pancreatitis in patients without a previous diagnosis of pancreatitis, or before exacerbation of the pancreatitis in patients having an existing pancreatitis condition.

### EXAMPLE

This Example shows that prophylactic administration of IL-10 decreases the severity of pancreatitis due to a pancreatitis-inducing event such as endoscopic retrograde cholangiopancreatography (ERCP). Prevention of post-ERCP pancreatitis in humans is a unique model to study the potential role of IL-10 in this setting.

### Patients and Methods

Over a 14-month period, 144 patients with definite indication for therapeutic ERCP were included in the study. Over the same period, a total of 1389 ERCPs were performed in the department, of which 752 were follow-up ERCPs for stent exchange and/or additional treatment of severe chronic pancreatitis. Of the 637 remaining patients, 204 were eligible for the study and 144 agreed to participate.

Eligible patients were those undergoing a first therapeutic ERCP. Exclusion criteria included previous therapeutic ERCP, less than 18 years of age, acute pancreatitis, hyperamylasemia or hyperlipasemia at baseline blood analysis, cholangitis (which might influence the cytokine plasma levels), chronic pancreatitis with calcifications and/or pancreatic duct dilation or marked parenchymal atrophy on computed tomographic scanning, known pancreatic cancer, ampulloma, metastatic tumors, severe systemic disease with possible pancreatic involvement, pain graded greater than 2 on a 1-10 visual scale before the procedure, pregnancy or breast feeding in women as well as use of inadequate contraception, use of any immunomodulating therapy within 90 days of entry, and use of any experimental drug within 30 days of entry.

Study Design

This single-center, double-blind, placebo-controlled randomized study compared three groups of patients who received a single intravenous (IV) injection of either placebo (group 0) or 4 μg/kg (group 1) or 20 μg/kg (group 2) recombinant human IL-10 thirty minutes before the therapeutic ERCP procedure. Drugs and placebos were packaged by Schering-Plough Research Institute (Kenilworth, N.J.) on the basis of a computer-generated random code (randomization by blocks of six). None of the physicians or nurses caring for the patients, running the study, or performing the procedures were aware of the codes or of the medications received.

The number of patients to be included in each group (at least 42) was calculated to meet the primary endpoint of the study (reduction of hyperhydrolasemia from 50% to 25%; a error, 0.05; β error, 0.2).

All patients were questioned about their clinical history, underwent a physical examination, and were asked to grade their pain on a 1-10 scale before and after therapeutic ERCP. They agreed to give blood immediately before the procedure (approximately 1 hour) and 4, 24, and 48 hours after the procedure to measure levels of hydrolases, C-reactive protein (CRP), IL-6, IL-8, and tumor necrosis factor (TNF).

Only the patients with a decision of therapeutic ERCP made before the procedure were considered, and all patients stayed overnight after the examination as a policy of the institution. Patients undergoing therapeutic ERCP during hospitalization for another reason were not included in the study. After discharge, the subjects were monitored as outpatients for the 48-hour blood sampling and clinical examination. Patients were sedated or anesthetized as usual (midazolam or propofol), avoiding morphine-like analgesics. They received antibiotic prophylaxis when they had cholestasis at the pretherapeutic work-up [Byl et al., Clin Infect Dis 20:1236-1240 (1995)], and no other treatment possibly influencing the occurrence of post-ERCP pancreatitis was given. No “protective” pancreatic stenting [Tarnasky et al., Gastroenterology 115:1518-1524 (1998)] in high-risk patients was performed.

The diagnosis and the type of treatment applied (biliary and/or pancreatic sphincterotomy, dilation, and/or stenting) were recorded. The whole duration of the procedure was mentioned for each case, and the operator was asked to evaluate the difficulty of the procedure on a 1-6 scale. All the procedures were performed by experienced endoscopists performing more than 200 ERCPs/year. Radiographs were carefully analyzed for the presence of pancreatic acinarization. The occurrence of immediate complication was recorded, as well as the use of coagulation for mild, non-clinically relevant bleeding occurring during the procedure. Pain was again graded on a 1 -10 scale at 4, 24, and 48 hours. Patients who had clinical pancreatitis at day 1 were systematically kept one additional night in the hospital. On day 7, clinical evaluation was obtained by a telephone call, and the patient was seen in outpatient visit, if appropriate. The complications were graded as mild, moderate, or severe according to the method of Cotton et al. [Cotton et al., Gastrointest. Endosc. 37:383-393 (1991)].

The primary endpoint of the study was the effect of IL-10 on serum levels of amylases and lipases, i.e., decrease in the incidence of hyperhydrolasemia, measured 4, 24, and 48 hours after ERCP. Secondary endpoints included incidence of clinical acute pancreatitis and evaluation of increases in IL-6, IL-8, and TNF serum levels in all patient groups and among patients with hyperhydrolasemia.

Cytokine Measurements

Cytokine plasma levels were measured using a commercially available enzyme-linked immunoassay (Medgenix, Fleurus, Belgium). Detection limits of the tests were 13, 5, and 10 pg/mL for IL-6, IL-8, and TNF, respectively. Amylases (normal value, <200 IU) and lipases (normal value, 200 IU) were measured at 30° C. using commercially available kits (Boehringer Mannheim, Germany) according to methods recommended by the International Federation of Clinical Chemistry.

Definitions

Hyperhydrolasemia was defined as an increase in amylase and/or lipase levels more than three times the normal values occurring within two days after the ERCP. Clinical pancreatitis was defined as hyperhydrolasemia (amylase and/or lipase levels greater than three times normal) associated with new or worsened abdominal pain persisting more than 4 hours after ERCP (≧3 grades on a 0-10 scale). Clinical pancreatitis was classified as mild when the length of hospital stay was less than three nights, moderate when the hospital stay was four to ten nights, and severe if more than ten days of hospitalization, intensive care unit (ICU) admission, or surgery was needed [Cotton et al., Gastrointest Endosc 37:383-393 (1991)].

Statistical Analysis

Results are expressed as means ±SEM. Comparisons between groups were done using the Pearson χ² test for categorical variables (or the Fisher exact test if appropriate) and 1-way analysis of variance (ANOVA) for continuous variables with Bonferroni correction as a post hoc test.

Potential risk factors for clinical pancreatitis or hyperhydrolasemia (as dependent variables) after therapeutic ERCP were included in a backward, stepwise multiple logistic regression to identify independent risk factors affecting the dependent variables (SPSS for windows, release 8.02, Chicago, Ill.). A P value of <0.05 was considered significant.

### Results

One hundred forty-four patients were included in the study, seven of whom were excluded [one case of preexisting unrecognized acute pancreatitis, one case of ampulloma, and five cases of failure to reach the papilla requiring percutaneous transhepatic access 2 Billroth II with a long afferent loop, 1 total gastrectomy with roux-en-Y anastomosis on two duodenal stenoses]. There were 45, 48, and 44 patients remaining in group 0, group 1, and group 2, respectively.

The three groups were comparable for age, sex, underlying disease, indication for treatment, type of treatment, and plasma levels of C-reactive protein (CRP), cytokines, and hydrolases at baseline (Table 1). Patients with suspected common bile duct stones are those with high clinical suspicion of stone migration for whom a decision of endoscopic sphincterotomy has been made. The early chronic pancreatitis group includes patients with acute relapsing pancreatitis in whom mild [Axon et al., Gut 10:1107-1112 (1984)] changes of chronic pancreatitis have been shown at secretin magnetic resonance cholangiopancreatography [Matos et al., Radiology 203:435-441 (1997)].

TABLE 1
Age, Sex, and Indications for Therapeutic ERCP

| | Group 0 | Group 1 | Group 2 |
|---|---|---|---|
| Diagnosis before the procedure | (n = 45) | (n = 48) | (n = 44) |
| Sex (M/F) | 17/28 | 24/24 | 18/26 |
| Age (yr)^{a} | 59 ± 2 | 64 ± 2 | 59 ± 2 |
| CBD stone (n) | 16 | 19 | 12 |
| Suspected CBD stone (n) | 5 | 5 | 5 |
| Biliary cancer (n) | 4 | 5 | 7 |
| Early chronic pancreatitis (n) | 5 | 4 | 6 |
| Pancreatic cyst (n) | 2 | 0 | 1 |
| Suspected pancreatic cancer (n) | 4 | 4 | 3 |
| Intraductal mucus-producing tumor (n) | 1 | 3 | 2 |
| Benign CBD lesion (n) | 3 | 3 | 4 |
| Choledococele (n) | 1 | 1 | 1 |
| Suspected SOD (n) | 4 | 4 | 3 |

^{a}Mean ± SEM.

TABLE 2
Therapeutic Maneuvers and Observations During Treatment

| | Group 0 | Group 1 | Group 2 | |
|---|---|---|---|---|
| | (n = 45) | (n = 48) | (n = 44) | P |
| Biliary sphincterotomy | 34 | 40 | 33 | 0.553 |
| Pancreatic sphincterotomy | 13 | 7 | 5 | 0.073 |
| Biliary dilation | 7 | 13 | 15 | 0.128 |
| Pancreatic dilation | 0 | 1 | 2 | 0.341 |
| Biliary stent | 5 | 10 | 13 | 0.097 |
| Pancreatic Stent | 0 | 1 | 2 | 0.341 |
| Precut | 5 | 0 | 3 | 0.070 |
| Acinarization | 5 | 4 | 5 | 0.866 |
| Use of coagulation | 8 | 5 | 7 | 0.577 |
| Duration of ERCP (min)^{a} | 47 ± 4 | 43 ± 3 | 55 ± 6 | 0.162 |
| Ease (score)^{a} | 3.1 ± 0.21 | 2.5 ± 0.2 | 2.9 ± 0.3 | 0.245 |

^{a}Mean ± SEM.

The group with suspected pancreatic cancer includes patients without radiologic evidence of tumors in whom tissue sampling from the pancreas and/or the bile duct was indicated. Intraductal mucinous papillary tumors were diagnosed by ERCP, pancreatoscopy, and/or tissue sampling into the main pancreatic duct.

All the patients included in the analysis had at least an opacification of the CBD (n=127) or of the pancreatic duct (n=111). Six patients (2 in each group) had only an opacification for failure of deep cannulation (n=3) or decision of the operator, during the procedure, to cancel or postpone the indication for treatment (n=3).

Therapeutic maneuvers are reported in Table 2. There were no significant differences between the three groups, although the number of endoscopic pancreatic sphincterotomies (EPSs) was slightly higher in group 0 than in groups 1 or 2, and the number of precuts was higher in groups 0 and 2 than in group 1. Other therapeutic observations which may influence the incidence of pancreatitis are discussed in Table 2.

In addition to pancreatitis, three other complications occurred, including two mild bleeding episodes, without the need for transfusion, and one retroperitoneal perforation treated conservatively and requiring hospitalization for seven days.

No adverse events related to administration of IL-10 were observed. No patient died, required surgery, or was admitted to the ICU because of complications of ERCP. Two cases of pancreatitis were classified as severe; both were observed in the placebo group. Computed tomographic scanning (CT) three and four days after ERCP in these two patients showed Balthazar grade D pancreatitis [Balthazar et al., Radiology 156:767-772 (1985)] in both cases (presence of a pancreatic collection that became infected in 1 case). No mortality related to ERCP was observed.

Plasma Levels of Hydrolases, CRP, and Cytokines After ERCP

No significant difference was observed in plasma levels of amylases, lipases, C-reactive protein (CRP), IL-6, IL-8, and TNF between the three groups (Table 3). Forty-three patients (31.4%) developed hyperhydrolasemia after ERCP (18 in group 0, 14 in group 1, and 11 in group 2; P=0.297) with no statistical difference in incidence between the three groups (Table 4). When only these patients were considered, TNF plasma levels were higher in group 0 than in groups 1 and 2 at 4 and 24 hours (18.8±2.5 vs. 12.6±1.2 and 12.6±1.4 pg/mL, P=0.038; and 15.2±1.2 vs. 11.4±0.8 and 11.5±0.9 pg/mL, P=0.016), respectively, and no difference was observed at baseline (12.2±0.9 vs. 10.7±0.7 and 12.2±0.9 pg/mL, P=0.41).

Clinical Post-ERCP Pancreatitis

Forty-one patients (29.9%) developed abdominal pain after the procedure. Among them, 19 patients (13.8%) had pain and hyperhydrolasemia and were hospitalized at least one additional night. The incidence was significantly higher in the placebo group than in groups 1 and 2 (P=0.038). Nineteen patients developed acute clinical pancreatitis (eleven in group 0, five in group 1, three in group 2; P=0.038), and two episodes of severe pancreatitis were observed in group 0 and none in groups 1 and 2 (Table 4). IL-6 serum levels measured 24 hours after ERCP, which are excellent markers of post-ERCP pancreatitis [Messmann et al., Gut 40:80-85 (1997)], were significantly higher in patients with acute pancreatitis (n=19) than in the control group (n=105), from which patients with acute cholangitis were excluded (90±27 vs. 47±6 pg/mL; P=0.02). Among patients with post-ERCP acute pancreatitis, IL-6 serum levels at 24 hours increased with severity (53±13, 68±25, and 340±174 pg/mL in mild, moderate, and severe cases, respectively; P<0.01).

Independent Factors Affecting Onset of Hyperhydrolasemia and Clinical Pancreatitis

In univariate analysis, in addition to the dose of IL-10 (P=0.016), the following variables were identified as risk factors for acute clinical pancreatitis with a P value of <0.2: EPS (P=0.008), acinarization (P=0.027), suspected SOD (P=0.181), and age (P=0.049) (Table 5).

Because the placebo group included more EPS than the two other groups, logistic regression analysis was performed with clinical pancreatitis as a dependent variable and the five risk factors identified above as independent variables to determine whether IL-10 pretreatment independently affected the occurrence of acute pancreatitis. Three variables were identified as independently influencing the occurrence of acute clinical pancreatitis in post-ERCP patients (see Table 5): pancreatic sphincterotomy (odds ratio [OR], 5.04; 95% confidence interval [95% Cl], 1.53-16.61; P=0.008), acinarization (OR, 8.19; 95% Cl, 1.83-36.57; P=0.006), and dose of IL-10 (OR, 0.46; 95% Cl, 0.22-0.96; P=0.039). The fact that IL-10 keeps its independent protective effect in the multivariate analysis is related to the decreased incidence of acute pancreatitis that is also observed in the subgroups of high-risk patients undergoing EPS or in whom acinarization was observed. Because of the small number of outcomes (n=19) and the number of independent variables (n=5), the results of the multivariate analysis should be taken with some reserve.

TABLE 3
Hydrolases, CRP, IL-6, IL-8, and TNF Serum Levels

| | Group 0 | Group 1 | Group 2 | |
|---|---|---|---|---|
| | (n = 45) | (n = 48) | (n = 44) | P |
| Amylases (IU/mL) | | | | |
| 0 | 114 ± 10 | 133 ± 9 | 119 ± 10 | 0.33 |
| 4 | 519 ± 102 | 430 ± 75 | 423 ± 105 | 0.72 |
| 24 | 930 ± 221 | 719 ± 176 | 498 ± 105 | 0.24 |
| 48 | 398 ± 100 | 504 ± 157 | 244 ± 61 | 0.30 |
| Lipases (IU/mL) | | | | |
| 0 | 82 ± 16 | 111 ± 15 | 121 ± 39 | 0.53 |
| 4 | 1238 ± 378 | 1259 ± 485 | 1771 ± 773 | 0.75 |
| 24 | 1264 ± 341 | 1612 ± 528 | 856 ± 355 | 0.44 |
| 48 | 361 ± 87 | 875 ± 353 | 404 ± 212 | 0.26 |
| CRP (mg/dL) | | | | |
| 0 | 2.6 ± 0.9 | 2.4 ± 0.7 | 2.2 ± 0.5 | 0.92 |
| 4 | 3.0 ± 1.1 | 2.3 ± 0.7 | 2.0 ± 0.4 | 0.65 |
| 24 | 3.9 ± 1.0 | 2.9 ± 0.6 | 3.0 ± 0.6 | 0.62 |
| 48 | 5.9 ± 1.8 | 4.1 ± 0.7 | 5.0 ± 1.1 | 0.27 |
| IL-6 (pg/mL) | | | | |
| 0 | 40 ± 12 | 55 ± 23 | 34 ± 4 | 0.59 |
| 4 | 91 ± 36 | 60 ± 17 | 45 ± 5 | 0.36 |
| 24 | 64 ± 14 | 78 ± 23 | 40 ± 5 | 0.26 |
| 48 | 66 ± 23 | 72 ± 22 | 41 ± 6 | 0.47 |
| IL-8 (pg/mL) | | | | |
| 0 | 34 ± 8 | 41 ± 8 | 50 ± 14 | 0.54 |
| 4 | 52 ± 14 | 39 ± 7 | 49 ± 12 | 0.66 |
| 24 | 36 ± 6 | 32 ± 6 | 48 ± 11 | 0.35 |
| 48 | 33 ± 7 | 37 ± 9 | 35 ± 10 | 0.96 |
| TNF (pg/mL) | | | | |
| 0 | 11 ± 1 | 11 ± 1 | 11 ± 1 | 0.74 |
| 4 | 15.9 ± 1.4 | 13.8 ± 0.9 | 12.1 ± 0.7 | 0.06 |
| 24 | 13.2 ± 0.8 | 14.0 ± 1.0 | 12.0 ± 0.7 | 0.23 |
| 48 | 11 ± 1 | 11 ± 1 | 11 ± 1 | 0.98 |

NOTE:
Data represent mean ± SEM

TABLE 4
Incidence of Hyperhydrolasemia, Postprocedural Abdominal Pain, and
Clinical Pancreatitis in the 3 Groups

| | Group 0 | Group 1 | Group 2 | |
|---|---|---|---|---|
| | (n = 45) | (n = 48) | (n = 44) | P |
| Pain (n) | 18 | 12 | 11 | 0.206 |
| Hyperhydrolasemia (n) | 18 | 14 | 11 | 0.297 |
| Pancreatitis | | | | |
| All (n) | 11^{a} | 5 | 3 | 0.038 |
| Mild (n) | 6 | 3 | 1 | |
| Moderate (n) | 3 | 2 | 2 | |
| Severe (n) | 2 | 0 | 0 | |
| Duration of stay for | 1.86 ± 0.80 | 0.46 ± 0.23 | 0.36 ± 0.22 | 0.060 |
| pancreatitis (days/ | | | | |
| # of patients treated) | | | | |

^{a}P = 0.046 and P = 0.017 for group 0 vs. group 1 and group 0 vs. group 2, respectively (Fisher exact test).

TABLE 5
Risk Factors for Clinical Pancreatitis After Therapeutic ERCP

| | Clinical | | | | |
|---|---|---|---|---|---|
| | pancreatitis | All | P | | |
| Risk Factor | (n = 19) | (n = 137) | (univariate) | Adjusted OR (95% Cl) | P |
| Significant in multivariate Analysis | | | | | |
| Pancreatic sphincterotomy (n) | 8 | 25^{b} | 0.008 | 5.04 (1.53-16.61) | 0.008 |
| Acinarization (n) | 5 | 14^{c} | 0.027 | 8.19 (1.83-36.57) | 0.006 |
| Dose of IL-10 (no. of patients in | 11/5/3 | 45/48/44 | 0.016 | 0.46 (0.22-0.96) | 0.039 |
| groups 0/1/2) | | | | | |

Significant in univariate analysis

| | | | |
|---|---|---|---|
| Age (yr)^{a} | 54.3 ± 3.3 | 60.8 ± 1.3 | 0.049 |
| Not significant | | | |
| Precut (n) | 1 | 8 | 0.69 |
| Suspected | | | |
| SOD (n) | 3 | 11 | 0.18 |
| Ease (score)^{a} | 3.2 ± 0.4 | 2.8 ± 0.1 | 0.22 |
| Duration (min)^{a} | 51 ± 7 | 48 ± 3 | 0.63 |
| Female sex (n) | 13 | 78 | 0.20 |
| Biliary phincterotomy (n) | 14 | 107 | 0.40 |

^{a}Mean ± SEM.
^{b}Among the 25 patients who underwent pancreatic sphincterotomy, the incidence of clinical pancreatitis was 5/13, 2/7, and 1/5 in groups 0, 1, and 2, respectively.
^{c}Among the 14 patients in whom an acinarization was observed, the incidence of acute pancreatitis was 4/5, 1/4, and 0/5 in groups 0, 1, and 2, respectively.

When ascertaining risk factors for hyperhydrolasemia, only EPS (P=0.046) and acinarization (P=0.070) were identified in univariate analysis. In a logistic regression analysis with hyperhydrolasemia as dependent factors, there were also only two independent factors affecting the development of hyperhydrolasemia (EPS: OR, 2.36; 95% Cl, 0.96-5.79; P=0.062; acinarization: OR, 2.78; 95% Cl, 0.86-8.99; P=0.088).

This Example shows that a single IV injection of IL-10 is safe and able to decrease the incidence of acute pancreatitis after therapeutic ERCP independently of any other risk factors associated with the procedure. This reduction of incidence averages 50%, and thus this treatment will have the major advantage of being applicable to outpatient therapy, at a limited cost.

All citations herein are incorporated herein by reference to the same extent as if each individual publication or patent application was specifically and individually indicated to be incorporated by reference.

Many modifications and variations of this invention can be made without departing from its spirit and scope, as will be apparent to those skilled in the art. The specific embodiments described herein are offered by way of example only, and the invention is to be limited by the terms of the appended claims, along with the full scope of equivalents to which such claims are entitled; and the invention is not to be limited by the specific embodiments that have been presented herein by way of example.

What is claimed is:

1. A method of preventing pancreatitis in a patient at risk of developing pancreatitis due to a pancreatitis-inducing event, comprising administering to the patient a therapeutically effective amount of IL-10.

2. The method of claim 1 wherein the IL-10 is administered within about 48 hours before or after the pancreatitis-inducing event.

3. The method of claim 1 wherein the pancreatitis-inducing event is endoscopic retrograde cholangiopancreatography.

4. The method of claim 1 wherein the IL-10 is parenterally administered.

5. The method of claim 1 wherein the therapeutically effective amount of IL-10 is from about 0.1 microgram to about 100 micrograms per kilogram of body weight.

6. The method of claim 1 wherein the amount is from about 2.5 micrograms to about 25 micrograms per kilogram of body weight.

7. The method of claim 1 wherein the IL-10 is pegylated.

8. A method of preventing exacerbation of pancreatitis in a patient at risk of a worsening pancreatitis condition due to a pancreatitis-inducing event, comprising administering to the patient a therapeutically effective amount of IL-10.

9. The method of claim 8 wherein the IL-10 is administered within about 48 hours before or after the pancreatitis-inducing event.

10. The method of claim 8 wherein the pancreatitis-inducing event is endoscopic retrograde cholangiopancreatography.

11. The method of claim 8 wherein the IL-10 is parenterally administered.

12. The method of claim 8 wherein the therapeutically effective amount of IL-10 is from about 0.1 microgram to about 100 micrograms per kilogram of body weight.

13. The method of claim 8 wherein the amount is from about 2.5 micrograms to about 25 micrograms per kilogram of body weight.

14. The method of claim 8 wherein the IL-10 is pegylated.

15. A method of preventing pancreatitis in a patient who is at risk for developing pancreatitis caused by endoscopic retrograde cholangiopancreatography (ERCP), comprising administering a therapeutically effective amount of IL-10 before the onset of the pancreatitis.

16. The method of claim 15 wherein the IL-10 is administered within about 48 hours before the ERCP.

17. The method of claim 15 wherein the IL-10 is administered at least 15 minutes before the ERCP.

18. The method of claim 15 wherein the amount of IL-10 is from about 1 to 50 μg/kg body weight.

19. The method of claim 15 wherein the administration is intravenous.

20. A method of preventing onset or exacerbation of pancreatitis in a patient, comprising administering IL-10 in an amount from about 1 to 50 μg/kg body weight, wherein the patient is subjected to:
biliary tract disease, alcohol, drug therapies (e.g., azathioprine, sulfasalazine, furosemide, valproic acid), vaccination against infectious disease, estrogen use, infection (e.g., mumps), hypertriglyceridemia, ERCP, percutaneous transhepatic cholangiography, structural abnormalities of the pancreatic duct (e.g., stricture, cancer, pancreas divisum), structural abnormalities of the common bile duct and ampullary region (e.g., choledochal cyst, sphincter of Oddi stenosis), hemobilia, bile duct obstruction, surgery (e.g., stomach, biliary tract and coronary artery bypass grafting), vascular disease (e.g. severe hypotension), blunt or penetrating trauma, hyperparathyroidism and hypercalcemia, renal transplantation, hereditary pancreatitis, peritoneal dialysis, or cigarette smoking.
